(19) United States
(12) Patent Application Publication
(10) Pub. No.: US 2026/0029418 A1
(43) Pub. Date: Jan. 29, 2026

(54) BIOMARKER FOR DIAGNOSING CENTRAL PRECOCIOUS PUBERTY IN FEMALES AND USES THEREOF

(71) Applicant: AFFILIATED HOSPITAL OF NANTONG UNIVERSITY, Nantong (CN)

(72) Inventors: Weixia YANG, Nantong (CN); Xinyi LU, Nantong (CN); Baolan SUN, Nantong (CN)

(21) Appl. No.: 19/349,975

(22) Filed: Oct. 4, 2025

Related U.S. Application Data
(63) Continuation of application No. PCT/CN2025/086092, filed on Mar. 31, 2025.

(30) Foreign Application Priority Data
Jul. 5, 2024 (CN) 202410904814.1

(51) Int. Cl. G01N 33/74; G01N 33/68

(52) U.S. Cl. CPC G01N 33/74; G01N 33/6893; G01N 2333/575; G01N 2800/04

(57) ABSTRACT

This application relates to the technical field of biomarkers, and in particular, to a biomarker for diagnosing central precocious puberty (CPP) in females and uses thereof. This application provides a biomarker for diagnosing CPP in females. The biomarker is one, two, or three of leptin, RBP4, or chemerin. Experiments performed in this application show that female children with CPP exhibit leptin, RBP4, and chemerin levels in peripheral blood significantly higher than those of healthy children, and that serum leptin and RBP4 levels in CPP patients change early during development of CPP, indicating that these three adipokines may participate in the development of CPP.

### TECHNICAL FIELD

This application relates to the technical field of biomarkers, and in particular, to a biomarker for diagnosing central precocious puberty (CPP) in females and uses thereof.

### BACKGROUND

CPP is a common pediatric endocrine disorder characterized by premature activation of the hypothalamic-pituitary-gonadal (HPG) axis, leading to rapid development of internal and external reproductive organs and the appearance of secondary sexual characteristics before the age of 7.5 years in girls and 9 years in boys. The incidence rate of the CPP is approximately 1 in 5000 to 1 in 10000, with girls being approximately 5 to 10 times more prone to be affected than boys. This CPP produces a significant impact on the grown-up height, psychological well-being, and other aspects of children. Premature menarche is considered a high-risk factor for breast cancer and is also associated with an increased mortality rate from hypertension, type-2 diabetes, stroke, estrogen-dependent cancers, and cardiovascular diseases. However, the pathogenesis of the CPP remains unclear. It is currently considered that factors such as genetics, nutrition, and environmental endocrine disruptors may contribute to the premature activation of the HPG axis. With rising living standards, the proportion of obese children is increasing annually. Early puberty is closely related to nutrition and body fat mass. Adipose tissue is an important endocrine organ, and can secrete a variety of bioactive mediators, known as adipokines, to play roles in endocrine metabolism and regulation. Therefore, obesity leads to dysregulation of adipokine secretion and then results in premature activation of the HPG axis, which may exert an important effect on the onset of the CPP.

Currently, a gold standard for diagnosing CPP is a GnRH stimulation test. However, this test is disadvantageously characterized by cumbersome operations, repeated blood sampling, and the risk that children with peripheral precocious puberty evolve to suffer CPP.

In recent years, both domestic and international studies have reported associations between the adipokine and the CPP. Leptin is the earliest studied, most extensively studied, and most thoroughly studied adipokine in CPP. Secreted by white adipose tissue, leptin serves functions of regulating appetite, energy expenditure, reproduction, and the like. The generation and secretion of leptin are positively correlated to body fat mass. An elevated serum leptin level has been observed in obese females and the females with CPP. In the hypothalamus, GnRH is secreted from GnRH neurons in a pulsatile manner, and acts on the pituitary to initiate the HPG axis and start puberty. A main regulatory center of the GnRH neurons is the hypothalamic neurons that express kisspeptin and neurokinin B. The leptin can cross the blood-brain barrier and bind to a leptin receptor (LepR) on a kisspeptin-expressing neuron, thereby stimulating the KISS-1 gene to overexpress the kisspeptin. At the pituitary level, highly concentrated serum leptin can also stimulate upregulation of GnRH receptor expression, increase the sensitivity of gonadotropins to GnRH pulses, and promote the connection between the hypothalamic GnRH neuron and the pituitary, thereby synchronizing the leptin-LH pulse with ovulation. Therefore, obesity leads to a large amount of fat accumulation in the body. Fat cells can produce and secrete leptin. However, obese individuals exhibit leptin resistance, which leads to hyperleptinemia. Leptin can cross the blood-brain barrier. When accumulating to a specified threshold, the leptin binds to the GnRH neurons on the hypothalamus and the LepR on the pituitary to exert a regulatory effect and cause premature activation of the HPG axis. This may be one of the pathogeneses of the CPP.

As primarily secreted by tissues such as white adipose tissue, the liver, and the placenta, Chemerin is a key regulatory factor of blood pressure, the immune system, angiogenesis, and inflammation, and is positively correlated to obesity and insulin resistance. Chemerin plays a role in regulating progesterone secretion in mouse follicles. In a PCOS rat model, chemerin can inhibit FSH-induced steroid hormone generation. In rat testicular Leydig cells, chemerin can inhibit hCG-stimulated testosterone (T) generation. In human primary granulosa cells cultured in vitro, chemerin can reduce IGF-1 induced secretion of progesterone and estradiol (E2). Due to the negative feedback mechanism of the HPG axis, a decreased sex hormone level can actuate a negative-feedback regulation to increase the secretion of GnRH, FSH, and LH in the hypothalamus and the pituitary. This may be one of the reasons for accelerating the HPG axis activation and leading to CPP in children.

RBP4 is primarily secreted by the liver and adipose tissue, and is responsible for binding and transporting retinol in the blood. The serum RBP4 level is positively correlated to visceral fat. Elevated serum RBP4 levels have been observed in adolescents undergoing normal pubertal development in a Korean population. In a population of healthy adult women in the United States, RBP4 is found to be significantly positively correlated to FSH and LH and negatively correlated to E2. It is found that in mouse ovaries, the FSH regulates the dynamic expression of RBP4 by affecting transcription factors HMGA1, SF-1, and LRH-1 through a cyclic AMP-protein kinase A (cAMP-PKA) signaling pathway. Furthermore, overexpression of RBP4 may induce insulin resistance in mice, inhibit the generation of SHBG in the liver, and increase the bioavailability of sex hormones. A decline in insulin sensitivity and a decrease in the SHBG level have also been observed in females with CPP. However, no studies have yet investigated the correlation between RBP4 and CPP either domestically or internationally so far.

### SUMMARY

An objective of this application is to provide a biomarker for diagnosing CPP in females and uses thereof. This application investigates the impact of adipokines on the HPG axis, and selects three adipokines that may be correlated to CPP: leptin, retinol binding protein 4 (RBP4), and chemerin, to test the concentrations of the adipokines leptin, RBP4, and chemerin in peripheral blood. Correlation analysis is performed with reference to clinical indicators of children with CPP, such as sex hormone levels, bone age, and BMI, so as to identify early warning indicators for CPP, especially the CPP in obese female children, and provide a basis for early diagnosis and prevention of CPP in females.

To achieve the foregoing objective, this application provides the following technical solutions:

This application provides a biomarker for diagnosing CPP in females. The biomarker is one, two, or three of leptin, RBP4, or chemerin.

This application discloses uses of the biomarker in preparing a diagnostic reagent for CPP in females.

This application provides a kit for testing CPP in females. The kit includes a reagent containing the biomarker.

Compared with the prior art, this application achieves at least the following beneficial effects:
1. Female children with CPP exhibit leptin, RBP4, and chemerin levels in peripheral blood significantly higher than those of healthy children, and serum leptin and RBP4 levels in CPP patients change early during development of CPP, indicating that these three adipokines may participate in the development of CPP.
2. Bone age, a difference between bone age and chronological age, testosterone (T), ultrasound-measured uterine length, and serum RBP4 level can be used as influencing factors and evaluation indicators for CPP in female children.
3. Combined testing of RBP4 and chemerin in peripheral blood of female children is of higher significance for diagnosing CPP than separate testing of leptin, RBP4, or chemerin, and exhibits potential clinical application significance.

### BRIEF DESCRIPTION OF DRAWINGS

FIG. 1(a) shows serum leptin levels of a CPP group versus a control group; FIG. 1(b) shows serum RBP4 levels of a CPP group versus a control group; FIG. 1(c) shows serum chemerin levels of a CPP group versus a control group (Note: * represents P less than 0.05, and ** represents P less than 0.01);

FIG. 2(a) shows serum leptin levels of an NM group versus an M group; FIG. 2(b) shows serum RBP4 levels of an NM group versus an M group; FIG. 2(c) shows serum chemerin levels of an NM group versus an M group (Note: * represents P less than 0.05, and ns represents P greater than 0.05);

FIG. 3 shows a heatmap illustrating correlation between leptin, chemerin, RBP4 and clinical data;

FIG. 4 shows a heatmap of analysis of correlation between leptin, chemerin, and RBP4;

FIG. 5 shows ROC curves of separate testing of leptin, chemerin, and RBP4; and

FIG. 6 shows an ROC curve of combined testing of RBP4 and chemerin.

### DETAILED DESCRIPTION

The technical solutions provided in this application are described in detail below with reference to exemplary embodiments, but the exemplary embodiments are not to be construed as limiting the scope of protection of this application.

### Experimental Example 1

### 1. Research Materials and Instruments

### 1.1 Research Subjects

(1) CPP group: 50 female children diagnosed with CPP in the Department of Pediatrics, Affiliated Hospital of Nantong University, from June 2021 to November 2022, were enrolled as research subjects, including 27 cases without menarche and 23 cases with menarche.

### Inclusion Criteria:

1. All diagnosed cases met the Consensus on the Diagnosis and Treatment of Central Precocious Puberty (2015):
1) Breast development occurs before the age of 8 years or menarche occurs before the age of 10.0 years in girls;
2) Gonadal enlargement occurs, that is, the B-ultrasound of the pelvic cavity shows that the uterine length is 3.4 to 4.0 cm, the ovarian volume is 1 to 3 mL (ovarian volume=length×width×thickness×0.5233), and a plurality of follicles with a diameter greater than or equal to 4 mm are observed, where a uterine body length greater than 3.2 cm is used as a cutoff value for diagnosing CPP;
3) Serum gonadotropins and sex hormones reach pubertal levels, with a basal LH value greater than 0.2 U/L; and activation of the hypothalamic-pituitary-gonadal (HPG) axis is indicated if the basal LH value is less than 0.2 U/L but an LH peak is greater than or equal to 5.0 U/L and an LH-to-FSH peak ratio is greater than or equal to 0.6 in a sex hormone stimulation test.
4) Bone age exceeds chronological age by 1 or more years; and
5) Linear growth is accelerated, with annual growth speed higher than that of healthy children of the same age.
2. The age at the time of diagnosis is less than 10 years.

### Exclusion Criteria:

1. Patients with precocious puberty secondary to the following diseases: congenital adrenal hyperplasia, McCune-Albright syndrome, or primary hypothyroidism;
2. Patients with intracranial lesions indicated by cranial imaging examination, such as hormonally active tumors or other space-occupying lesions; or patients with central nervous system abnormalities caused by acquired injuries, such as trauma, surgery, or radiotherapy or chemotherapy;
3. Patents with other congenital or hereditary diseases; and
4. Patents with other related endocrine diseases such as diabetes, hyperthyroidism, hypothyroidism, dwarfism, and gigantism.
(2) Control group: 50 healthy female children of the same age range who underwent puberty physical examinations in the pediatric outpatient clinic of this hospital were selected as the control group.

The investigation in this application was registered and approved by the Ethics Committee of the Affiliated Hospital of Nantong University with the ethics review number 2022-K028-01. All experimental subjects were informed of the relevant information and signed the informed consent.

### 2.1.2 Main Reagents and Instruments

TABLE 1
Main reagents

| Test indicator | Reagent name | Manufacturer |
|---|---|---|
| Leptin | ELISA kit | Shanghai Lianshuo Biological |
| | | Technology Co., Ltd. |
| Retinol binding | ELISA kit | Shanghai Lianshuo Biological |
| protein 4 (RBP4) | | Technology Co., Ltd. |
| Chemerin | ELISA kit | Shanghai Lianshuo Biological |
| | | Technology Co., Ltd. |

TABLE 2
Main instruments

| Instrument name | Manufacturer |
|---|---|
| Anxin centrifuge | Yancheng Anxin Experimental |
| | Instruments Co., Ltd. |
| Ultra-low temperature freezer | Qingdao Haier Co., Ltd. |
| (−80° C.) | |
| S1010E low-speed centrifuge | Scilogex, LLC |
| Constant temperature water bath | Shanghai Jinghong Experimental |
| | Equipment Co., Ltd. |
| BioTekELx808IU microplate reader | BioTek Instruments, Inc. |
| Pipette (10 μL, 100 μL, 1000 μL) | Eppendorf |
| centrifuge tube | NEST Biotechnology Co., Ltd. |

### 2. Experimental Procedure and Methods

### 2.1 Clinical Data and Blood Sampling

Detailed information about general condition and medical history of all patents was collected and recorded, including age, height, weight, calculated body mass index (BMI=weight in kg/height in m²), Tanner stage, and breast stage. The uterine and ovarian development of children patents was examined by using color Doppler ultrasound. The sex hormone panel (FSH, LH, progesterone, testosterone (T), estradiol (E2), and pituitary prolactin) was measured by the Clinical Biochemistry Laboratory of this hospital and collected through the HIS system. All pediatric patients underwent an examination of an X-ray anteroposterior radiograph of the left wrist, and the bone age was calculated according to the Bone Age Atlas (2016).

With informed consent, 5 ml of peripheral venous blood was collected from each child with CPP and each control group child in the fasting state in the early morning and placed in a procoagulant biochemical tube. The tube was centrifuged at 3500 rpm for 10 minutes. 200 μL of the supernatant serum was aliquoted with a pipette into 3 dry, sterile, and enzyme-free centrifuge tubes, and then sealed, labeled, and stored in a −80° C. freezer in the biological sample bank of this hospital.

### 2.2 Measurement of Leptin Concentration in Serum Samples by Means of ELISA

(1) The serum sample was taken out of the −80° C. freezer, and left to stand at room temperature until the sample dissolved. The ELISA kit was taken out of the 4° C. refrigerator and left to stand at room temperature for 30 minutes.
(2) Dilution of the standard: Five 1.5 mL centrifuge tubes are taken. 120 μL of standard diluent was added into each tube. 120 μL of stock standard was added into a first tube, and mixed well with a low-speed centrifuge to obtain a diluted standard. 120 μL of the diluted standard was drawn up from the first tube and transferred to a second tube with a pipette. This process went on so that the standard was diluted a plurality of times. Finally, the concentrations of the standards in the five tubes were 40 μg/L, 20 μg/L, 10 μg/L, 5 μg/L, and 2.5 μg/L, respectively.
(3) Sample addition: 1) Standard wells: with 50 μL of standard and 50 μL of streptavidin-HRP added; 2) blank wells: with nothing added; 3) sample wells: with 40 μL of sample, 10 μL of RBP4 antibody, and 50 μL of streptavidin-HRP added. The microplate with the wells was sealed with a sealing film and gently shaken thoroughly. The sample was incubated in a 37° C. water bath for 60 minutes.
(4) Preparing a washing solution: 30× concentrated washing solution was diluted 30-fold by using distilled water and set aside.
(5) Washing: The sealing film was removed, the liquid in the wells was discarded, the plate was spun dry, and each well was filled with washing solution, left to stand for 30 seconds, and then emptied of the washing solution. The above process was repeated 5 times, and the plate was blotted dry with filter paper.
(6) Color development: 50 μL of color developer A and 50 μL of color developer B were added to each well separately. The plate was covered with a new sealing film, gently shaken thoroughly, and placed in a 37° C. water bath in the dark for 15 minutes to develop color.
(7) Termination: 50 μL of stop solution was added into each well to terminate the reaction.
(8) Measurement: Within 10 minutes after adding the stop solution, the absorbance (OD value) of each well was measured sequentially at a wavelength of 450 nm, and the instrument was zeroed by using the blank wells.
(9) Calculation of concentration: A regression equation of the standard curve (R²>0.92) was calculated in an Excel spreadsheet based on the concentration of the standard and the corresponding optical density (OD) value. The leptin concentration of the corresponding sample was calculated in the regression equation based on the OD value of the sample.

### 2.3 Measurement of RBP4 Concentration in Serum Samples by Means of ELISA

(1) All serum samples were diluted at 1:5 with PBS before measurement. 10 μL of sample was added into 40 μL of PBS, and mixed well by using a low-speed centrifuge.
(2) The concentrations of the standards after dilution in five tubes were 40 μg/L, 20 μg/L, 10 μg/L, 5 μg/L, and 2.5 μg/L, respectively.

The remaining steps are the same as those of measuring leptin.

### 2.4 Measurement of Chemerin Concentration in Serum Samples by Means of ELISA

The measurement method is the same as that of RBP4.

The concentrations of the standards after dilution in five tubes were 48 ng/L, 24 ng/L, 12 ng/L, 6 ng/L, and 3 ng/L, respectively.

### 2.5 Statistical Analysis

Data collation and analysis were performed by using statistical software SPSS 24.0 and Graphpad Prism 9.3.0. The normality of all data was assessed by performing a Kolmogorov-Smirnov test. Normally distributed measurement data was expressed as mean±standard deviation (X̅±S), and two groups of samples were compared by using an independent-samples t-test. Non-normally distributed measurement data was expressed as median (P25, P75), and two groups of samples were compared by using a Mann-Whitney U test. Spearman correlation analysis was performed to analyze the relationships between leptin, RBP4, and chemerin, as well as between these three biomarkers and clinical data such as height, weight, BMI, sex hormones, bone age, the difference between bone age and chronological age, and uterine length. All statistical tests were performed at a significance level of α=0.05, and differences were considered statistically significant if the P-value is less than 0.05. Predictors of CPP in female children were analyzed by using logistic regression. The diagnostic significance of leptin, RBP4, and chemerin alone and in combination for CPP was analyzed by using a receiver operating characteristic (ROC) curve, and the diagnostic significance was evaluated by using indicators such as area under the curve (AUC), 95% confidence interval (CI), cut-off value, P value, sensitivity, and specificity.

### 3. Results

### 3.1 Comparison of Clinical Data Between the CPP Group and the Control Group

According to the diagnostic criteria set forth in the Consensus on Diagnosis and Treatment of Central Precocious Puberty (2015), a total of 50 CPP-group samples and 50 control-group samples were collected for this experiment.

Statistical analysis shows no statistically significant differences in age distribution, progesterone, or pituitary prolactin between the two groups (P>0.05). However, the CPP group exhibits higher levels of height, weight, BMI, Tanner stage, breast stage, bone age, difference between bone age and chronological age, FSH, LH, T, E2, and ultrasound-measured uterine length than the control group, and these indicators are of statistical significance (P<0.05), as shown in Table 3.

TABLE 3
Comparison of clinical data between the CPP group and the control group

| | CPP group (50 samples) | Control group (50 samples) | t/Z value | P-value |
|---|---|---|---|---|
| Age (year) | 8.53 ± 1.10 | 8.58 ± 0.97 | 0.205 | 0.838 |
| Height (m) | 1.40 ± 0.10 | 1.34 ± 0.07 | 3.160 | 0.002** |
| Weight (kg) | 36.42 ± 8.81 | 31.52 ± 6.92 | 3.094 | 0.003** |
| BMI (kg/m²) | 18.43 ± 2.77 | 17.24−2.40 | 2.298 | 0.024* |
| Tanner stage | 2(2, 3) | 2(1, 2) | −3.087 | 0.002** |
| Breast stage | 3(2, 4) | 2(2, 3) | −4.424 | <0.001** |
| Bone age (year) | 10.71(9.00, 12.42) | 9.00 (8.00, 9.42) | −4.320 | <0.001** |
| Difference between | 1.95 ± 1.23 | 0.35 ± 0.85 | 3.641 | <0.001** |
| bone age and | | | | |
| chronological age | | | | |
| (year) | | | | |
| FSH (mIU/ml) | 5.19 ± 1.79 | 2.95 ± 1.48 | 6.712 | <0.001** |
| LH (mIU/ml) | 1.96(0.80, 4.50) | 0.19(0.10, 0.42) | −6.378 | <0.001** |
| Progesterone (ng/ml) | 0.21(0.15, 0.48) | 0.23(0.13, 0.32) | −0.306 | 0.759 |
| T (ng/ml) | 0.20(0.10, 0.30) | 0.00(0.00, 0.20) | −3.217 | 0.001** |
| E2 (pg/ml) | 27.00(12.00, 43.00) | 9.50(5.00, 15.00) | −3.792 | <0.001** |
| Pituitary prolactin | 195.00(128.00, 294.00) | 149.50(114.00, 258.00) | −1.210 | 0.226 |
| (mIU/L) | | | | |
| Uterine length (mm) | 28.04 ± 7.65 | 20.12 ± 3.45 | 6.653 | <0.001** |

Note:
BMI represents body mass index, FSH represents follicle stimulating hormone, LH represents luteinizing hormone, T represents testosterone, E2 represents estradiol,
*indicates P < 0.05, and
**indicates P < 0.01.

### 3.2 Measurement and Comparison of Serum Leptin, RBP4, and Chemerin Levels of the CPP Group Versus the Control Group

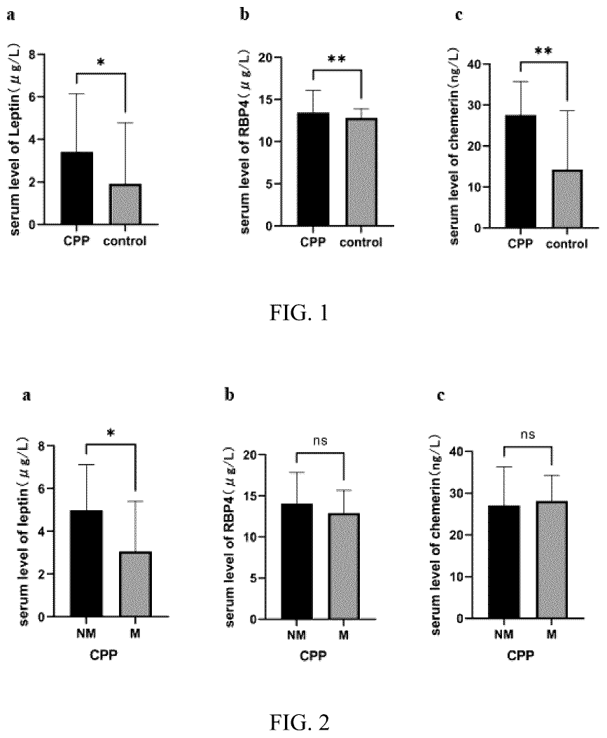

The serum Leptin, RBP4, and chemerin levels in all children were measured by means of ELISA. Statistical analysis revealed that the levels of these three factors in serum of the CPP group were significantly higher than those of the control group (P<0.05), and the differences are of statistical significance, as shown in Table 4 and FIG. 1.

TABLE 4
Comparison of serum leptin, RBP4, chemerin levels between two groups

| | CPP group (50 samples) | Control group (50 samples) | Z-value | P-value |
|---|---|---|---|---|
| Leptin (μg/L) | 3.41(1.41, 6.10) | 1.87(1.29, 4.58) | −2.437 | 0.015* |
| RBP4 (μg/L) | 13.44(12.61, 15.90) | 12.81(10.76, 13.86) | −2.616 | 0.009** |
| Chemerin (ng/L) | 27.61(10.94, 35.76) | 14.25(8.76, 27.66) | −3.202 | 0.001** |

Note:
*indicates P < 0.05, and
**indicates P < 0.01.

3.3 Comparison Between the NM Group and the M Group in Children with CPP

Based on clinical manifestations, the CPP group was divided into a non-menarche (NM) group and a menarche (M) group. Statistical analysis on the two groups shows that there are differences between the NM group and the M group in age distribution, height, weight, BMI, Tanner stage, breast stage, bone age, difference between bone age and chronological age, FSH, LH, T, E2, and uterine length measured with B-ultrasound, and the differences are of statistical significance (P<0.05). The serum leptin level of the NM group is higher in that of the M group, and the difference is of statistical significance (P<0.05). There are no statistically significant differences in RBP4 and chemerin levels between the two groups (P>0.05), as shown in Table 5 and FIG. 2.

TABLE 5
Comparison of clinical data and serum indicators between the NM group and the

| | CPP group (50 samples) | | | |
|---|---|---|---|---|
| | NM group (27 samples) | M group (23 samples) | t/Z value | P-value |
| Age (year) | 7.70 ± 0.77 | 9.52 ± 0.32 | −11.128 | <0.001** |
| Height (m) | 1.33 ± 0.08 | 1.48 ± 0.05 | −8.049 | <0.001** |
| Weight (kg) | 31.24 ± 6.53 | 42.50 ± 7.13 | −5.827 | <0.001** |
| BMI (kg/m²) | 17.57 ± 2.37 | 19.45 ± 2.91 | −2.510 | 0.015* |
| Tanner stage | 2(1, 2) | 4(3,4) | −5.323 | <0.001** |
| Breast stage | 2(2, 3) | 4(3,4) | −5.209 | <0.001** |
| Bone age (year) | 9.00(8.42, 9.00) | 12.42(12.00, 13.00) | −5.750 | <0.001** |
| Difference between bone age | 1.29 ± 1.03 | 2.73 ± 0.90 | −5.249 | <0.001** |
| and chronological age (year) | | | | |
| FSH (mIU/ml) | 4.31 ± 1.68 | 6.28 ± 1.45 | −4.690 | <0.001** |
| LH (mIU/ml) | 0.83 (0.40, 1.96) | 4.36(2.23, 6.71) | −4.292 | <0.001** |
| Progesterone (ng/ml) | 0.22 (0.15, 0.35) | 0.19(0.13, 0.59) | −0.487 | 0.627 |
| T (ng/ml) | 0.10 (0.00, 0.20) | 0.25(0.20, 0.30) | −3.015 | 0.003* |
| E2 (pg/ml) | 12.00(4.00, 25.00) | 41.00(30.00, 49.00) | −4.212 | <0.001** |
| Pituitary prolactin (mIU/L) | 156.00(126.00, 264.00) | 241.00(148.00, 318.00) | −1.335 | 0.182 |
| Uterine length (mm) | 22.92 ± 5.04 | 34.09 ± 5.48 | −7.355 | <0.001** |
| Leptin (μg/L) | 5.00(1.60, 6.99) | 3.07(1.35, 5.14) | −1.976 | 0.048* |
| RBP4 (μg/L) | 14.05(12.80, 16.89) | 12.90(12.02, 14.89) | −1.849 | 0.064 |
| Chemerin (ng/L) | 27.06(11.15, 36.31) | 28.16(10.44, 33.26) | −1.080 | 0.280 |

Note:
*indicates P < 0.05, and
**indicates P < 0.01.

### 3.4 Analysis of Correlation Between Serum Leptin, RBP4, and Chemerin Levels and Clinical Data

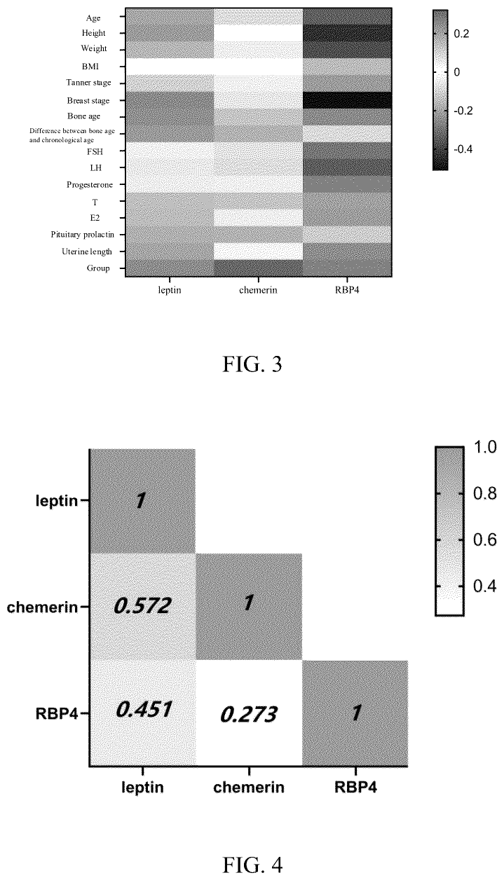

No significant correlation is exhibited between serum leptin and chemerin levels and clinical data (P>0.05). The serum RBP4 level is negatively correlated to age, height, weight, breast stage, FSH, and LH (P<0.05), as shown in Table 6 and FIG. 3. The serum leptin level is positively correlated to serum chemerin and RBP4 levels (P<0.05), as shown in Table 7 and FIG. 4.

TABLE 6
Analysis of correlation between serum leptin,
RBP4, and chemerin levels and clinical data

| | Leptin | | Chemerin | | RBP4 | |
|---|---|---|---|---|---|---|
| | r-value | P-value | r-value | P-value | r-value | P-value |
| Age | −0.196 | 0.173 | −0.084 | 0.561 | −0.344 | 0.015* |
| Height | −0.221 | 0.123 | −0.017 | 0.908 | −0.441 | 0.001** |
| Weight | −0.164 | 0.255 | −0.048 | 0.734 | −0.379 | 0.007** |
| BMI | −0.014 | 0.922 | −0.012 | 0.935 | −0.155 | 0.284 |
| Tanner stage | −0.108 | 0.454 | −0.043 | 0.765 | −0.218 | 0.129 |
| Breast stage | −0.256 | 0.073 | −0.065 | 0.652 | −0.508 | <0.001** |
| Bone age | −0.244 | 0.088 | −0.132 | 0.362 | −0.252 | 0.077 |
| Difference between | 0.216 | 0.132 | −0.172 | 0.232 | −0.088 | 0.543 |
| bone age and | | | | | | |
| chronological age | | | | | | |
| FSH | −0.043 | 0.767 | 0.067 | 0.646 | −0.298 | 0.036* |
| LH | −0.058 | 0.689 | 0.080 | 0.579 | −0.348 | 0.013* |
| Progesterone | −0.051 | 0.730 | −0.046 | 0.758 | −0.271 | 0.062 |
| T | 0.141 | 0.340 | 0.130 | 0.378 | −0.209 | 0.153 |
| E2 | −0.152 | 0.301 | −0.048 | 0.746 | −0.217 | 0.138 |
| Pituitary | −0.176 | 0.231 | 0.168 | 0.255 | −0.117 | 0.427 |
| prolactin | | | | | | |
| Uterine | −0.199 | 0.175 | 0.031 | 0.834 | −0.249 | 0.088 |
| length | | | | | | |
| Group | 0.245 | 0.014* | 0.322 | 0.001** | 0.263 | 0.008** |

Note:
*indicates P < 0.05, and **indicates P < 0.01.

TABLE 7
Analysis of correlation between leptin, chemerin, and RBP4

| | Leptin | | Chemerin | | RBP4 | |
|---|---|---|---|---|---|---|
| | r- | P- | r- | P- | r- | P- |
| | value | value | value | value | value | value |
| Leptin | 1 | | 0.572 | <0.001** | 0.451 | <0.001** |
| Chemerin | 0.572 | <0.001** | 1 | | 0.273 | 0.055 |
| RBP4 | 0.451 | <0.001** | 0.273 | 0.055 | 1 | |

Note:
**indicates P < 0.01.

### 3.5 Analysis of Influencing Factors and Evaluation Indicators for CPP in Female Children

Univariate analysis reveals that height, weight, BMI, Tanner stage, breast stage, bone age, difference between bone age and chronological age, FSH, LH, T, E2, ultrasound-measured uterine length, serum leptin level, serum chemerin level, and serum RBP4 level are all single factors that influence the occurrence of CPP in female children. Based on statistical principles, a multivariate logistic regression analysis was performed by using 11 statistically significant factors such as BMI, Tanner stage, breast stage, bone age, difference between bone age and chronological age, T, E2, ultrasound-measured uterine length, serum leptin level, serum chemerin level, and serum RBP4 level as independent variables. The results shows that the bone age, the difference between bone age and chronological age, T, ultrasound-measured uterine length, and serum RBP4 level can serve as influencing factors and evaluation indicators for CPP in female children, as shown in Table 8.

TABLE 8
Logistic regression analysis of factors inducing CPP in female children

| Factor | B | SE | P | OR | 95% CI |
|---|---|---|---|---|---|
| BMI (kg/m²) | −0.186 | 0.237 | 0.431 | 0.830 | (0.522 to 1.320) |
| Tanner stage | −0.228 | 0.616 | 0.711 | 0.796 | (0.238 to 2.661) |
| Breast stage | 0.558 | 0.902 | 0.536 | 1.748 | (0.298 to 10.240) |
| Bone age (year) | −3.041 | 1.065 | 0.004** | 0.048 | (0.006 to 0.386) |
| Difference between bone age | 2.742 | 0.820 | 0.001** | 15.518 | (3.108 to 77.472) |
| and chronological age (year) | | | | | |
| T (ng/ml) | 1.588 | 0.705 | 0.024* | 4.892 | (1.229 to 19.475) |
| E2 (pg/ml) | 0.059 | 0.032 | 0.068 | 1.061 | (0.996 to 1.130) |
| Uterine length (mm) | 0.602 | 0.227 | 0.008** | 1.826 | (1.170 to 2.849) |
| Leptin (μg/L) | 0.304 | 0.232 | 0.191 | 1.355 | (0.859 to 2.136) |
| RBP4 (μg/L) | 0.265 | 0.128 | 0.038* | 1.303 | (1.014 to 1.675) |
| Chemerin (ng/L) | −0.056 | 0.045 | 0.217 | 0.946 | (0.866 to 1.033) |

Note:
*indicates P < 0.05, and
**indicates P < 0.01.

### 3.6 Diagnostic Significance of Serum Leptin, Chemerin, and RBP4 Levels in Diagnosing CPP in Female Children

Leptin, chemerin, and RBP4 each exert effects on different parts of the hypothalamic-pituitary-gonadal (HPG) axis, and therefore, may play a role in the initiation of the HPG axis, and may provide important guidance for the early clinical diagnosis and prevention of CPP. This application investigates the diagnostic significance of leptin, chemerin, and RBP4 levels separately in diagnosing CPP in female children, as well as the diagnostic significance of the three biomarkers in combination in diagnosing CPP in female children.

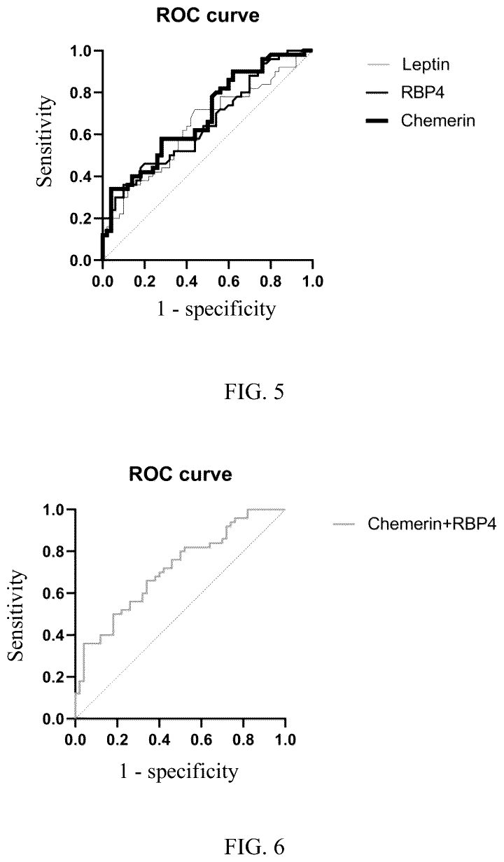

The area under the curve (AUC) of RBP4 is slightly higher than that of leptin while the AUC of chemerin is slightly higher than that of both RBP4 and leptin. The sensitivity of leptin is 72.0%, the specificity of leptin is 58.0%, the sensitivity of RBP4 is 46.0%, the specificity of RBP4 is 80.0%, the sensitivity of chemerin is 58.0%, and the specificity of chemerin is 72.0%. The AUC of RBP4 and chemerin tested in combination is 0.716, with a sensitivity of 66.0% and a specificity of 66.0%, indicating higher diagnostic significance than the biomarkers tested separately. The ROC curves are shown in FIG. 5 and FIG. 6, and the related data of the ROC curves is shown in Table 9.

TABLE 9
Relevant indicators of ROC

| | | Sensi- | Speci- | | | | |
|---|---|---|---|---|---|---|---|
| | Cut-off | tivity | ficity | Youden | | 95% | |
| | value | (%) | (%) | index | AUC | CI | P |
| Leptin | 2.069 | 72.0 | 58.0 | 0.300 | 0.641 | 0.533 to 0.750 | 0.015 |
| RBP4 | 13.987 | 46.0 | 80.0 | 0.260 | 0.652 | 0.545 to 0.758 | 0.009 |
| Chemerin | 22.663 | 58.0 | 72.0 | 0.300 | 0.686 | 0.583 to 0.789 | 0.001 |
| RBP4 + | 0.524 | 66.0 | 66.0 | 0.320 | 0.716 | 0.616 to 0.815 | <0.001 |
| Chemerin | | | | | | | |

In this application, by analyzing and comparing the clinical data of female children with CPP versus age-matched healthy controls, it is found that the height, weight, and bone age of the CPP group are significantly higher than those of the control group (P<0.05). This indicates that the children with CPP exhibit an obviously accelerated growth and development speed compared with the age-matched healthy controls. Due to the rapid skeletal growth, bone age is much greater than the chronological age, leading to premature closure of epiphyseal plates, thereby affecting the final grown-up height of the pediatric patients. On the other hand, the levels of T and E2 in the CPP group are also higher than those in the control group (P<0.05), indicating a risk of developing PCOS and estrogen-dependent diseases. Therefore, early diagnosis and prevention of CPP are of great importance. In addition, the BMI of the CPP group is significantly higher than that of the control group (P<0.05). Within the CPP group, the BMI of the children with menarche (M group) is also significantly higher than that of the children without menarche (NM group) (P<0.05), indicating a link between obesity and the occurrence and progression of CPP.

In this application, the serum leptin level of the CPP group is higher than that of the control group (P<0.05). Meanwhile, the serum leptin level of the NM group is higher than that of the M group (P<0.05). Because the serum sOB-R levels were not measured in the experiments, it cannot be excluded that a decline in free serum leptin level in the M group is caused by binding between leptin and sOB-R in the serum.

In this application, correlation analysis reveals that leptin is correlated to RBP4 and chemerin separately. In addition, the research herein further indicates that the serum leptin level is positively correlated to serum chemerin level (r=0.572, P<0.001). Both leptin and chemerin in serum are secreted by adipose tissue, and may regulate each other.

RBP4 is mainly secreted by the liver and adipose tissue. In this application, the serum RBP4 levels in the CPP group are significantly higher than those in the normal control group (P<0.05). Correlation analysis shows that the serum RBP4 level in the pediatric patients with CPP are negatively correlated to age, height, weight, breast developmental stage, FSH, and LH.

Our research results in this application, for the first time, reveals that the expression of the adipokines RBP4 and chemerin is also upregulated in the serum of female pediatric patients with CPP. In addition, a significant elevation of the serum leptin and RBP4 levels can be observed in the early stage of CPP, and the correlation analysis indicates that leptin may affect the expression levels of RBP4 and chemerin. Furthermore, logistic regression analysis reveals that the bone age, the difference between bone age and chronological age, testosterone (T), the ultrasound-measured uterine length, and the serum RBP4 level may serve as predictors of CPP in female children. In this application, the above indicators used in combination may facilitate early diagnosis of CPP in female children. This application indicates a correlation between leptin, RBP4, and chemerin. Accordingly, we investigated the diagnostic significance of leptin, RBP4, and chemerin tested separately, as well as the diagnostic significance of RBP4 and chemerin tested in combination in females with CPP. Based on the area under the curve (AUC), the diagnostic significance of RBP4 and chemerin tested in combination is higher than that of the three biomarkers tested separately. The diagnostic significance is considerable, and this application provides a new, relatively fast and convenient method for the early diagnosis of female children with CPP.

The above embodiments are merely preferred embodiments of this application.

What is claimed is:

1. Uses of RBP4 and chemerin as biomarkers in preparing a diagnostic reagent for central precocious puberty in females.
